(19) United States
(12) Patent Application Publication
(10) Pub. No.: US 2019/0038624 A1
(43) Pub. Date: Feb. 7, 2019

(54) USE OF ABELSON TYROSINE KINASE INHIBITOR IN TREATMENT OF MULTIPLE SYSTEM ATROPHY

(71) Applicants: Daniel Dung Truong, Huntington Beach, CA (US); Trang Nguyen Ngoc Vo, Ho Chi Minh City (VN); Cong Huu Nguyen, Ho Chi Minh City (VN)

(72) Inventors: Daniel Dung Truong, Huntington Beach, CA (US); Trang Nguyen Ngoc Vo, Ho Chi Minh City (VN); Cong Huu Nguyen, Ho Chi Minh City (VN)

(21) Appl. No.: 16/053,800

(22) Filed: Aug. 3, 2018

Related U.S. Application Data
(60) Provisional application No. 62/541,052, filed on Aug. 3, 2017.

(51) Int. Cl. A61K 31/506; A61P 25/16; A61P 25/28; A61K 9/00

(52) U.S. Cl. CPC A61K 31/506; A61P 25/16; A61K 9/4866; A61K 9/0053; A61P 25/28

(57) ABSTRACT

The present disclosure relates to a method for treating or alleviating a symptom of MSA, comprising administering a therapeutically effective amount of an abelson (Abl) tyrosine kinase inhibitor to a subject in need thereof.

### CROSS REFERENCE TO RELATED APPLICATIONS

This application claims priority to, and is a non-provisional application of, U.S. Provisional Patent application No. 62/541,052, filed on Aug. 3, 2017, now pending, which is hereby incorporated by reference in its entirety.

### FIELD OF THE DISCLOSURE

The present disclosure relates to the field of medicine, more specifically the disclosure is directed towards the treatment, delay and amelioration of multiple system atrophy (MSA).

### BACKGROUND OF THE DISCLOSURE

MSA, also known as Shy-Drager syndrome, is a rare neurodegenerative disorder characterized by tremors, slow movement, muscle rigidity, and postural instability (collectively known as parkinsonism) due to dysfunction of the autonomic nervous system, and ataxia. Current therapy depends on symptomatic management, and no treatment has yet been reported to prolong survival or reverse disease progression of MSA.

### SUMMARY OF THE DISCLOSURE

The present disclosure provides a method for treating or alleviating a symptom of MSA, comprising administering a therapeutically effective amount of an abelson (Abl) tyrosine kinase inhibitor to a subject in need thereof.

Certain embodiments of the symptom of MSA includes orthostatic hypotension, impotence, loss of sweating, dry mouth and urinary retention, incontinence, tremor, muscular rigidity, hypokinesia, impaired balance, impaired speech, impaired swallowing and ataxia. Certain embodiments of the MSA includes parkinsonian subtype, cerebellar dysfunction subtype and Shy-Drager syndrome.

Certain embodiments of the Abl tyrosine kinase inhibitor includes imatinib, gefitinib, erlotinib dasatinib, sunitinib, adavosertib, lapatinib and nilotinib.

The embodiments of the administration of the Abl tyrosine kinase inhibitor include the administration of about 100 to about 300 mg (preferably about 200 mg) per day for 14 to 30 weeks (preferably 24 weeks).

The embodiment of the administration of the Abl tyrosine kinase is oral or injection.

### BRIEF DESCRIPTION OF THE DRAWING

FIG. 1 shows cerebral MRI demonstrates brainstem and cerebellar atrophy, as well as the “hot cross bun” sign.

FIG. 2 shows a significant decline of 6.8 and 2.2 points in the UMSARS and UMSARS-II, respectively, compared to baseline.

### DETAILED DESCRIPTION OF THE DISCLOSURE

Terms not specifically defined herein should be given the meanings that would be given to them by one of skill in the art in light of the disclosure and the context. As used in the specification, however, unless specified to the contrary, the following terms have the meaning indicated and the following conventions are adhered to.

The use of the word “a” or “an” in the claims and/or the specification may mean “one,” but it is also consistent with the meaning of “one or more,” “at least one,” and “one or more than one.”

The terms “treatment” and “treating” embrace both preventative, i.e. prophylactic, or therapeutic, i.e. curative and/or palliative, treatment. Thus the terms “treatment” and “treating” comprise therapeutic treatment of patients having already developed said condition, in particular in manifest form. Therapeutic treatment may be symptomatic treatment in order to relieve the symptoms of the specific indication or causal treatment in order to reverse or partially reverse the conditions of the indication or to stop or slow down progression of the disease. Thus the compositions and methods of the present disclosure may be used for instance as therapeutic treatment over a period of time as well as for chronic therapy. In addition the terms “treatment” and “treating” comprise prophylactic treatment, i.e. a treatment of patients at risk to develop a condition mentioned hereinbefore, thus reducing said risk.

The term “therapeutically effective amount” means an amount of a compound of the present disclosure that (i) treats or prevents the particular disease or condition, (ii) attenuates, ameliorates, or eliminates one or more symptoms of the particular disease or condition, or (iii) prevents or delays the onset of one or more symptoms of the particular disease or condition described herein.

In one aspect, the present disclosure provides a method for treating or alleviating a symptom of MSA, comprising administering a therapeutically effective amount of an abelson (Abl) tyrosine kinase inhibitor to a subject in need thereof.

In some embodiments, the symptom of MSA includes, but is not limited to, orthostatic hypotension, impotence, loss of sweating, dry mouth and urinary retention, incontinence, tremor, muscular rigidity, hypokinesia, impaired balance, impaired speech, impaired swallowing and ataxia.

In some embodiments, the MSA includes, but is not limited to, parkinsonian subtype, cerebellar dysfunction subtype and Shy-Drager syndrome.

In some embodiments, the Abl tyrosine kinase inhibitor includes, but is not limited to, imatinib, gefitinib, erlotinib dasatinib, sunitinib, adavosertib, lapatinib and nilotinib.

The dose range of the Abl tyrosine kinase inhibitor applicable per day is usually from about 100 to about 300 mg, preferably from about 150 to 250 mg. preferably, the dose is about 200 mg per day. In one embodiment, the Abl tyrosine kinase inhibitor is administered for around 14 to 30 weeks, preferably, around 24 weeks.

The actual therapeutically effective amount or therapeutic dosage will of course depend on factors known by those skilled in the art such as age and weight of the patient, route of administration and severity of disease. In any case the combination will be administered at dosages and in a manner which allows a therapeutically effective amount to be delivered based upon subject's unique condition.

For oral administration, suitable pharmaceutical compositions of the disclosure include powders, granules, pills, tablets, lozenges, chews, gels, and capsules as well as liquids, syrups, suspensions, elixirs, and emulsions. These compositions may also include anti-oxidants, flavorants, preservatives, suspending, thickening and emulsifying agents, colorants, flavoring agents and other pharmaceutically acceptable additives. Formulations for oral administration may be formulated to be immediate release or modified release, where modified release includes delayed, sustained, pulsed, controlled, targeted and programmed release.

For parenteral administration, the Abl tyrosine kinase inhibitor is administered directly into the blood stream, into muscle, or into an internal organ via an intravenous, intraarterial, intraperitoneal, intramuscular, subcutaneous or other injection or infusion. Parenteral formulations may be prepared in aqueous injection solutions which may contain, in addition to the Abl tyrosine kinase inhibitor of the disclosure, buffers, antioxidants, bacteriostats, salts, carbohydrates, and other additives commonly employed in such solutions. Parenteral administrations may be immediate release or modified release (such as an injected or implanted depot).

The Abl tyrosine kinase inhibitor of the present disclosure may also be administered topically, (intra)dermally, or transdermally to the skin or mucosa. Typical formulations include gels, hydrogels, lotions, solutions, creams, ointments, dressings, foams, skin patches, wafers, implants and microemulsions. The Abl tyrosine kinase inhibitor may also be administered via inhalation or intanasal administration, such as with a dry powder, an aerosol spray or as drops. Additional routes of administration for compounds of the present disclosure include intravaginal and rectal (by means of a suppository, pessary or enema), and ocular and aural.

It should be understood that many additional changes in the details, materials, steps and arrangements of parts, which have been herein described and illustrated in order to explain the nature of the present disclosure, may be made by those skilled in the art while still remaining within the principles and scope of the disclosure.

### EXAMPLES

### Example 1 Multiple System Atrophy Treated with an Abelson Tyrosine Kinase Inhibitor

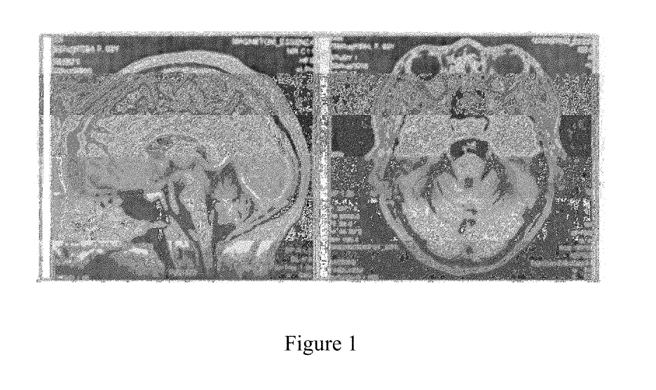

A 62-year-old Vietnamese woman who presented with inability to walk. The disease started 4 years ago with a slowing of her gait and tremors at rest in both hands. The patient initially was diagnosed with PD, but she did not respond to dopaminergic therapy. She had been experiencing dizziness, dysphagia during the past year, and urinary urgency during the previous six months. Her brain MRI reveals brainstem and cerebellar atrophy, with the characteristic “hot cross bun” sign in the axial section (see FIG. 1). Finally, the patient was diagnosed with “probable multiple system atrophy” according to the 2008 MSA consensus statement.

Upon admission, the patient could not walk without assistance and needed intermittent bladder catheterization because of urinary incontinence. The Unified MSA Rating Scale (UMSARS) score was 56 points, and the UMSARS motor subsection part II (UMSARS-II) score was 25 points. Autonomic testing demonstrated severe dysfunction of the sympathetic and parasympathetic nervous systems, with significant orthostatic hypotension and poor heart rate variability during change of posture, deep breathing, and the Valsalva test. Sympathetic skin response was also absent on the soles and palms. Informed consent was obtained from the patient and her family before treatment of oral Nilotinib 200 mg every 4 weeks. The monthly monitoring included a physical and a neurological exam, an electrocardiogram, and complete blood count and chemistry.

By the end of the 24-week study, the patient experienced some nonserious adverse events (AEs) that did not require hospitalization, including mild skin irritation, dizziness, and mild headache. All of these AEs resolved with continuous use of the drug. The nonserious AEs (pneumonia, cold virus, mild back pain, mild headache, mild dysgraphia, mild hallucination, skin irritation, dizziness, and transient QTc prolongation on electrocardiograms) appeared to be similar to those reported by Pagan et al. in 20167 in the first study of Nilotinib in human neurodegenerative diseases.

The patient's clinical outcome improved progressively. She could walk slowly without assistance at the end of the study. There were decreases of 11 and 6 points on the UMSARS and UMSARS-II, respectively, at week 24 compared with baseline. The patient also experienced significant improvement of non-motor symptoms, such as swallowing, constipation, and urinary function. Although we used a different dose of Nilotinib, our MSA patient responded to the drug in a manner similar to the PD and DLB subjects in the Pagan et al. study. They had a decline of 11.1 points in their UPDRS I-IV during 24 weeks of treatment with 300 mg Nilotinib. The significant improvement of our patient's UMSARS suggests that Nilotinib could produce beneficial clinical effects in MSA patients.

The main limitation of this study was our inability to measure the pharmacokinetics of Nilotinib in our patient, including Nilotinib levels in cerebrospinal fluid (CSF) and CSF disease-related biomarkers such as α-synuclein and homovanillic acid-dopamine metabolite because necessary materials were not available in our country. Additionally, this is a descriptive study of one case report with a short follow-up period.

This case report appears to be the first case of MSA treated with an Abelson tyrosine kinase inhibitor. Our data suggest that Nilotinib 200 mg once daily may be safe and effective for this MSA patient.

### Example 2 MSA and Abelson Tyrosine Kinase Inhibitor

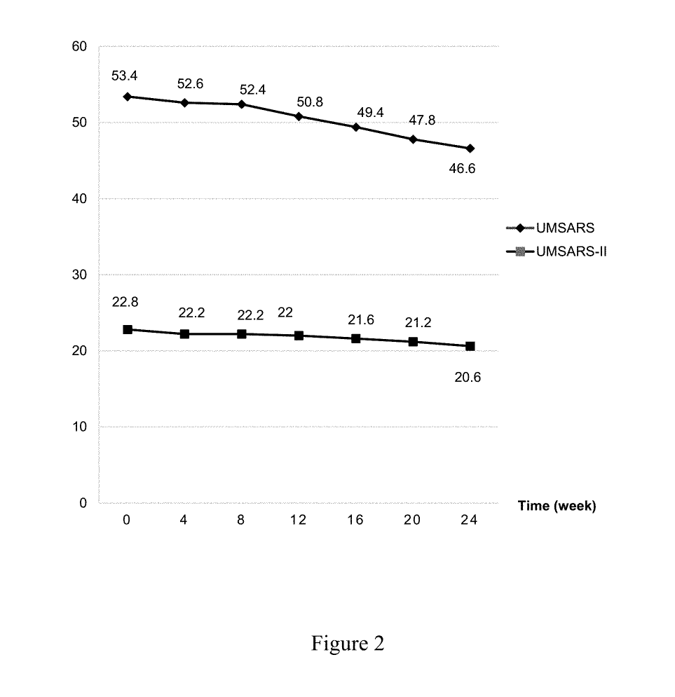

Five patients fulfilled the consensus criteria for “probable MSA” and received oral Nilotinib 200 mg daily for 24 weeks. Disease progression was monitored every 4 weeks by measuring the patient's motor and non-motor symptoms with the Unified Multiple System Atrophy Rating Scale (UMSARS). Mini Mental State Examination (MMSE) was used to screen for cognitive impairment. Although there were no serious adverse events (AEs) during the drug administration, the patients experienced some non-serious AEs, including mild skin irritation, dizziness, mild headache, and mild blurry vision. However, these AEs resolved with continued administration of the drug. By the end of 24 weeks, the patient showed improvement of non-motor symptoms, such as swallowing, constipation, and urinary function. Furthermore, there was a significant decline of 6.8 and 2.2 points in the UMSARS and UMSARS-II, respectively, compared to baseline (see FIG. 2). MMSE results were normal throughout treatment. Our study results suggest that Nilotinib 200 mg once daily may be safe and effective for this MSA patient.

| | Age | Gender | Adverse events |
|---|---|---|---|
| Patient 1 | 62 | F | Mild headache, dizziness, mild skin irritation |
| Patient 2 | 67 | M | None |
| Patient 3 | 48 | M | Mild headache, mild blurry vision |
| Patient 4 | 62 | F | None |
| Patient 5 | 65 | F | Mild skin irritation |

UMSARS

| | | Week | Week | Week | Week | Week | Week |
|---|---|---|---|---|---|---|---|
| | Basline | 4 | 8 | 12 | 16 | 20 | 24 |
| Patient 1 | 56 | 55 | 55 | 51 | 48 | 46 | 44 |
| Patient 2 | 42 | 41 | 41 | 39 | 39 | 36 | 36 |
| Patient 3 | 44 | 42 | 42 | 40 | 39 | 38 | 37 |
| Patient 4 | 81 | 81 | 81 | 81 | 81 | 79 | 79 |
| Patient 5 | 44 | 44 | 43 | 43 | 40 | 40 | 37 |
| Average | 53.4 | 52.6 | 52.4 | 50.8 | 49.4 | 47.8 | 46.6 |

UMSARS-II

| | | Week | Week | Week | Week | Week | Week |
|---|---|---|---|---|---|---|---|
| | Basline | 4 | 8 | 12 | 16 | 20 | 24 |
| Patient 1 | 25 | 23 | 23 | 22 | 21 | 20 | 19 |
| Patient 2 | 15 | 15 | 15 | 14 | 14 | 13 | 13 |
| Patient 3 | 18 | 17 | 17 | 18 | 18 | 18 | 17 |
| Patient 4 | 38 | 38 | 38 | 38 | 38 | 38 | 38 |
| Patient 5 | 18 | 18 | 18 | 18 | 17 | 17 | 16 |
| Average | 22.8 | 22.2 | 22.2 | 22 | 21.6 | 21.2 | 20.6 |

What is claimed is:

1. A method for treating or alleviating a symptom of MSA, comprising administering a therapeutically effective amount of an abelson (Abl) tyrosine kinase inhibitor to a subject in need thereof.

2. The method of claim 1, wherein the symptom of MSA is orthostatic hypotension, impotence, loss of sweating, dry mouth and urinary retention, incontinence, tremor, muscular rigidity, hypokinesia, impaired balance, impaired speech, impaired swallowing or ataxia.

3. The method of claim 1, wherein the MSA is parkinsonian subtype, cerebellar dysfunction subtype or Shy-Drager syndrome.

4. The method of claim 1, wherein the Abl tyrosine kinase is imatinib, gefitinib, erlotinib dasatinib, sunitinib, adavosertib, lapatinib or nilotinib.

5. The method of claim 1, wherein the Abl tyrosine kinase is nilotinib.

6. The method of claim 1, wherein the Abl tyrosine kinase inhibitor applicable per day is from about 100 to about 300 mg.

7. The method of claim 1, wherein the Abl tyrosine kinase inhibitor applicable per day is from about 200 mg.

8. The method of claim 1, wherein the Abl tyrosine kinase inhibitor is administered for around 14 to 30 weeks.

9. The method of claim 1, wherein the Abl tyrosine kinase inhibitor is administered for around 24 weeks.

10. The method of claim 1, wherein the Abl tyrosine kinase inhibitor is administered with a dose of about 100 to about 300 mg per day for around 14 to 30 weeks.

11. The method of claim 1, wherein the Abl tyrosine kinase inhibitor is administered with a dose of about 200 mg per day for around 24 weeks.

12. The method of claim 11, wherein the administration is oral.
